(19) United States
(12) Patent Application Publication
(10) Pub. No.: US 2024/0261128 A1
(43) Pub. Date: Aug. 8, 2024

(54) knob adjustable elastic wrist guard

(71) Applicant: Dongguan Fushun Sports Goods Co., Ltd., Dongguan (CN)

(72) Inventor: Xiaogao Gu, Ji'an (CN)

(21) Appl. No.: 18/358,136

(22) Filed: Jul. 25, 2023

(30) Foreign Application Priority Data
Feb. 7, 2023 (CN) 202320142145X

(51) Int. Cl. A61F 5/058

(52) U.S. Cl. CPC A61F 5/05866; A61F 2005/0197

(57) ABSTRACT

A knob adjustable elastic wrist guard is disclosed, including a guard body and a knob adjustable elastic assembly. The guard body consists of a sheath, a bib portion and an elastic band, the bib portion is configured to locate at a middle of a rear side of the sheath, the elastic band is configured to detachably thread on the bib portion. A threading buckle is provided at a left end of the elastic band, and the knob adjustable elastic assembly is configured to locate at a front side of the sheath. The wrist guard of the present disclosure can be used for left and right hands, and the tightness can be adjusted by the knob, which is convenient and comfortable to wear.

### TECHNICAL FIELD

This application belongs to the technical field of wrist guard, specifically relate to a new knob adjustable elastic wrist guard.

### BACKGROUND

Wrist fractures, distal radius/ulnar fractures, soft tissue injuries to the wrist, carpal tunnel syndrome (mouse hand), wrist arthritis, tendonitis and tenosynovitis, during postoperative rehabilitation after removal of cast or splint, it is often necessary to add a wrist brace to protect the wrist and allow for better recovery.

A general protector must have a certain tightening force in order to achieve a corresponding protective effect, each person's ability to withstand elastic is different, and feelings after wearing the protector are different. However an existing protector is not convenient to adjust the elastic, and after adjustment may not achieve a comfortable elasticity, thus affecting the recovery from injury. Therefore, the present disclosure provides a new knob adjustable elastic wrist guard, to address above proposed technical problems.

### SUMMARY

The present disclosure provides a new knob adjustable elastic wrist guard to address above proposed problems, the wrist guard can be used for left and right hands, easy to adjust elasticity by using the knob, and comfortable to wear.

In some embodiments, the knob adjustable elastic wrist guard includes a guard body and a knob adjustable elastic assembly. The guard body consists of a sheath, a bib portion and an elastic band, the bib portion is configured to locate at a middle of a rear side of the sheath, the elastic band is configured to detachably thread on the bib portion, a threading buckle is provided at a left end of the elastic band, and the knob adjustable elastic assembly is configured to locate at a front side of the sheath. The knob adjustable elastic assembly includes a knob, threading lugs and a traction wire, the knob is configured to set on a right side surface of the sheath, the threading lugs are configured to uniformly set at four corners of the front side of the sheath, the traction wire is configured to wind on the knob, and the traction wire is configured to cross threading the threading lugs and the threading buckle.

Preferably, in some embodiments, donning holes are provided on a rear side of the sheath, and the donning holes are configured to locate on two top sides of the rear side of the sheath.

Preferably, in some embodiments, there are four sets of the threading lugs.

Preferably, in some embodiments, the bib portion is configured as an I-shape construction.

Preferably, in some embodiments, the sheath is made of an antibacterial elastic fabric.

The present disclosure adopts above structures to achieve beneficial effect as follows. The new knob adjustable elastic wrist guard provided by the present disclosure is set by providing the knob adjustable elastic assembly, when turning the knob, the knob makes the traction wire wind up or loosen, thus adjusting the tension force of the traction wire on the elastic band, and meanwhile the traction wire wind up and loosens on the sheath, similar to the principle of tying a shoelace. The wrist guard of the present disclosure can be used for left and right hands, easy to adjust elasticity by using the knob, and comfortable to wear.

### BRIEF DESCRIPTION OF THE DRAWINGS

FIG. 1 shows a front view of the knob adjustable elastic wrist guard in accordance with some embodiments of the present disclosure.

FIG. 2 shows a rear view of the knob adjustable elastic wrist guard in accordance with some embodiments of the present disclosure.

In the drawings, the reference signs are as follows. 1. guard body, 2. knob adjustable elastic assembly, 3. sheath, 4. bib portion, 5. elastic band, 6. threading buckle, 7. knob, 8. threading lugs, 9. traction wire, 10. donning holes.

### DETAILED DESCRIPTION OF THE EMBODIMENTS

The technical solutions of the present disclosure will be clearly and completely described below in conjunction with the accompanying drawings, and it is clear that the described embodiments are a part of the embodiments of the present disclosure, and not all of them. Based on the embodiments in the present disclosure, all other embodiments obtained by a person of ordinary sill in the art without making creative labor fall within the scope of protection of the present disclosure.

In the description of the present disclosure, it should be noted that the terms “center”, “top”, “bottom”, “left”, “right”, “vertical”, “horizontal”, “inner”, “outer”, etc. indicating the orientation or positional relationships are based on the orientation or positional relationships shown in the accompanying drawings, and are intended only to facilitate and simplify the description of the present disclosure, not to indicate or imply that the device or element referred to must have a particular orientation, be constructed and operate in a particular orientation, and therefore are not to be construed as limiting the present disclosure. Furthermore, the terms “first”, “second”, and “third” are used for descriptive purposes only and are not to be construed as indicating or implying relative importance. The present disclosure is described in further detail below in conjunction with the accompanying drawings.

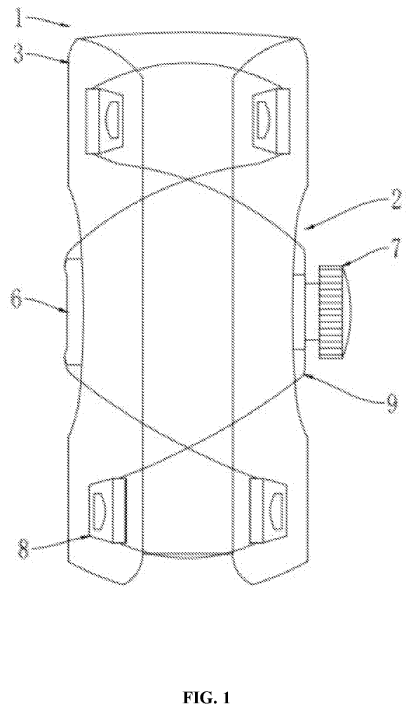

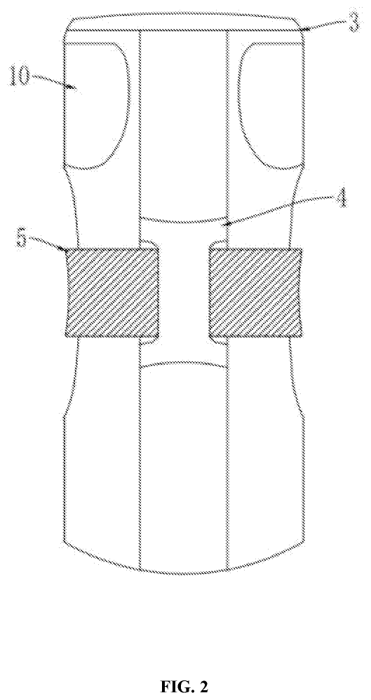

As shown in FIG. 1 and FIG. 2, in some embodiments, the present disclosure provides a new knob adjustable elastic wrist guard, including a guard body (1) and a knob adjustable elastic assembly (2). The guard body (1) consists of a sheath (3), a bib portion (4) and an elastic band (5), the bib portion (4) is configured to locate at a middle of a rear side of the sheath (3), the bib portion (4) is configured as an I-shape construction, and the elastic band (5) is configured to detachably thread on the bib portion (4). A threading buckle (6) is provided at a left end of the elastic band (5), and the knob adjustable elastic assembly (2) is configured to locate at a front side of the sheath (3). The knob adjustable elastic assembly (2) includes a knob (7), threading lugs (8) and a traction wire (9), the knob (7) is configured to set on a right side surface of the sheath (3), the threading lugs (8) are configured to uniformly set at four corners of the front side of the sheath (3), and there are four sets of the threading lugs (8). The traction wire (9) is configured to wind on the knob (7), and the traction wire (9) is configured to cross threading the threading lugs (8) and the threading buckle (6). When the knob 7 is turned, the knob 7 makes the traction wire 9 wind up or loosen, thus adjusting the tightness of the traction wire 9 on the elastic band 5, and meanwhile the traction wire 9 tightens and loosen the sheath 3, similar to the principle of tying shoelaces. The wrist guard can be used for both left and right hands, by using the knob 7 to adjust the tightness, the tightness is easy to adjust and the wrist guard is comfortable to wear.

In some embodiments, donning holes (10) are provided on a rear side of the sheath (3), and the donning holes (10) are configured to locate on two top sides of the rear side of the sheath (3). By such designs, it is more ergonomic and comfortable to wear. In some embodiments, the sheath 3 is made of an antibacterial elastic fabric.

In specific use, the sheath 3 is worn at left or right wrist, and when the knob 7 is turned, the knob 7 causes the traction wire 9 to wind up, thus adjusting the tightness of the traction wire 9 on the elastic band 5. And the traction wire 9 simultaneously tightens the sheath 3 similar to the principle of lacing a shoe, and adjusts the guard body 1 to a suitable tightness.

The above description of the present disclosure and its embodiments is not limiting, and what is shown in the accompanying drawing is only one of the embodiments of the present disclosure, and an actual structure is not limited to it. All in all if a person of ordinary skill in the art is inspired by it, without departing from the creation purpose of the present disclosure, and designing without creatively a structural way and embodiment similar to this technical solution, all shall fall within the scope of protection of the application.

What is claimed is:

1. A knob adjustable elastic wrist guard, comprising a guard body (1) and a knob adjustable elastic assembly (2); wherein
the guard body (1) consists of a sheath (3), a bib portion (4) and an elastic band (5), the bib portion (4) is configured to locate at a middle of a rear side of the sheath (3), the elastic band (5) is configured to detachably thread on the bib portion (4), a threading buckle (6) is provided at a left end of the elastic band (5), and the knob adjustable elastic assembly (2) is configured to locate at a front side of the sheath (3);
the knob adjustable elastic assembly (2) comprises a knob (7), threading lugs (8) and a traction wire (9), the knob (7) is configured to set on a right side surface of the sheath (3), the threading lugs (8) are configured to uniformly set at four corners of the front side of the sheath (3), the traction wire (9) is configured to wind on the knob (7), and the traction wire (9) is configured to cross threading the threading lugs (8) and the threading buckle (6).

2. The knob adjustable elastic wrist guard according to claim 1, wherein donning holes (10) are provided on a rear side of the sheath (3), and the donning holes (10) are configured to locate on two top sides of the rear side of the sheath (3).

3. The knob adjustable elastic wrist guard according to claim 2, wherein there are four sets of the threading lugs (8).

4. The knob adjustable elastic wrist guard according to claim 3, wherein the bib portion (4) is configured as an I-shape construction.

5. The knob adjustable elastic wrist guard according to claim 4, wherein the sheath (3) is made of an antibacterial elastic fabric.
